(19) United States
(12) Patent Application Publication
(10) Pub. No.: US 2024/0400506 A1
(43) Pub. Date: Dec. 5, 2024

(54) DIROXIMEL FUMARATE PARTICLES HAVING IMPROVED FLOW PROPERTIES AND METHODS OF MAKING SAME

(71) Applicant: BIOGEN MA INC., Cambridge, MA (US)

(72) Inventors: Liang Chen, Cambridge, MA (US); Erwin Irdam, Melrose, MA (US); Daw-long Albert Kwok, Cambridge, MA (US); Nocole Madden, Cambridge, MA (US); John Mascho, Cambridge, MA (US)

(21) Appl. No.: 18/692,541

(22) PCT Filed: Sep. 16, 2022
(86) PCT No.: PCT/US22/43837

§ 371 Date: Mar. 15, 2024

Related U.S. Application Data
(60) Provisional application No. 63/357,130, filed on Jun. 30, 2022.
(60) Provisional application No. 63/245,476, filed on Sep. 17, 2021.

(51) Int. Cl. C07D 207/404; A61K 9/20; A61K 31/4015

(52) U.S. Cl. CPC C07D 207/404; A61K 9/2009; A61K 9/2027; A61K 9/2054; A61K 31/4015

(57) ABSTRACT

Many active pharmaceutical ingredients (API) are available in tablet form. Particles of the API should have a favorable compaction profile in order to be compressed into tablets, especially when the API is present in a large weight percentage in the tablet. Disclosed herein are particles of diroximel fumarate having improved characteristic for tablet formation, and methods of producing the same.

### RELATED APPLICATIONS

This application claims the benefit of U.S. Provisional Application Ser. No. 63/245,476 filed Sep. 17, 2021 and U.S. Provisional Application Ser. No. 63/357,130 filed Jun. 30, 2022, the entire teachings of which are incorporated herein by reference.

### BACKGROUND

Diroximel fumarate is sold under the brand name Vumerity and is a medication used for the treatment of relapsing forms of multiple sclerosis. Diroximel fumarate was first disclosed in U.S. Pat. No. 8,669,281 and approved for medical use in the United States in October 2019.

Delivering active pharmaceutical ingredients (APIs) as a tablet is highly desirable because of ease of administration and convenience to the patient. Tablets are formed by compressing powder blends containing the API particles and additives. To successfully prepare tablet formulations, the API particles and/or blends comprising API particles and additives used to form the tablet must have a number of specific properties, including good flow, compaction, and mechanical properties. Preparing tablets can be particularly difficult when there is a high drug load, e.g., in tablets comprising greater than 60% by weight of API.

### SUMMARY

It has now been found that standard particle milling processes, such as dry milling, produce diroximel fumarate particles having less desirable properties for tablet formation. It has also been found that diroximel fumarate particles produced by a wet milling process have significantly improved mechanical and flow properties compared to particles prepared by the dry milling process. For example, the tablet hardness after compaction of wet milled diroximel fumarate is more uniform than after compaction of dry milled diroximel fumarate (see Example 5). This benefits the tablet coating process as well as the dissolution behavior. In addition, the flow function coefficients used to quantify flow properties for neat wet milled diroximel fumarate particles are higher compared to neat dry milled diroximel fumarate particles (see Example 3). This higher flow function coefficient indicates that wet milled diroximel fumarate has superior flow properties compared with dry milled diroximel fumarate. Cohesion of the disclosed particles is lower than that of dry milled diroximel fumarate particles (See Example 3). When cohesion is lower, the particles flow more smoothly and have better properties for storage. Diroximel fumarate particles having superior flow and compaction properties are characterized by less variability in size, as measured by the particle size span, and by a reduction in the D₁₀, i.e., the value under which 10% of the diroximel fumarate particles have a smaller volume diameter. They are also characterized by increased smoothness. The disclosed diroximel fumarate particles have been found to have improved dissolution properties, which is advantageous for this API (see Example 7). Based on these discoveries, diroximel fumarate particles having superior flow and compaction properties, methods of preparing the diroximel particles with superior flow and compaction properties, and blends comprising the diroximel fumarate particles having superior flow and compaction properties, and tablets comprising the diroximel fumarate particles having more uniform hardness are disclosed herein.

In one embodiment, the invention is diroximel fumarate particles having a span of 1.1 to 2.5, a D₁₀ of 20-50 μm, a D₅₀ of 70-130 μm, and a D₉₀ of 150-250 μm.

Another embodiment of the invention is a blend comprising the disclosed diroximel fumarate particles and one or more additives, fillers and/or excipients. In one aspect, the blend comprises from 60-92.5% wt. % diroximel fumarate particles based on the total weight of the blend.

Another embodiment of the invention is a tablet comprising the disclosed diroximel fumarate particles or the disclosed blends.

Another embodiment of the invention is a method of producing the disclosed diroximel fumarate particles. The particles are suitable for use in high load tablets. The method comprises subjecting a slurry of pre-milled diroximel fumarate particles in a solvent to a wet milling step to produce milled diroximel fumarate particles; and subjecting the milled diroximel fumarate particles to a ripening step to produce the diroximel fumarate particles.

Yet another embodiment of the invention is diroximel fumarate particles prepared by the disclosed methods.

### BRIEF DESCRIPTION OF THE FIGURES

FIG. 1 shows Scanning Electron Micrograph images of dry milled diroximel fumarate particles and the disclosed diroximel fumarate particles.

FIG. 2 is a bar graph of flow function coefficient of neat dry milled diroximel fumarate particles compared to neat disclosed diroximel fumarate particles.

FIG. 3 is a bar graph of cohesion of neat dry milled diroximel fumarate particles compared to the neat disclosed diroximel fumarate particles.

FIG. 4 is a bar graph of flow function coefficient of blends comprising dry milled diroximel fumarate particles compared to blends comprising the disclosed diroximel fumarate particles.

FIG. 5 is a graph of Hardness vs. Compaction Pressure for tables made from dry milled diroximel fumarate particles and the disclosed diroximel fumarate particles.

FIG. 6 is a graph showing a comparison dissolution profiles of tablets made from both dry milled API (Blue diamond, square, and triangle plots) and wet milled API (red square plot-leftmost curve).

### DETAILED DESCRIPTION

Disclosed herein are diroximel fumarate particles with good mechanical properties for tablet formation, such as high flowability, consistent compactability, and low cohesion. The disclosed diroximel fumarate particles are characterized by a reduced particle size distribution, e.g., diminished variation in size among the particles and/or by a reduced number of small particles.

Particle size is expressed in terms of volume diameter, which is used to provide a dimensional description of particles which are non-spherical. “Volume diameter” refers to the diameter of a sphere with equal volume of the non-spherical particle. Particle size can be measured according to methods well known in the art, including a laser diffraction technique that correlates light scattering to particle volume on which effective length or effective diameter is calculated.

Particle size distribution is expressed in terms of D₅₀, D₁₀, D₉₀ and span. “Span” is a measure for particle size distribution. It is calculated according to the following equation:

$$Span = ( {D}_{90} - {D}_{10} ) / {D}_{50}$$

A lower span value means that the particles are more consistently sized, and that there is a reduced particle size variation.

“D₅₀”, also known as the median diameter, corresponds to the value under which 50% of the particles has a lower volume diameter. “D₉₀” corresponds to the value under which 90% of the particles has a lower volume diameter. “D₁₀” corresponds to the value under which 10% of the particles has a lower volume diameter.

The disclosed diroximel fumarate particles are characterized by a reduced span, indicating that the particles in a sample are more consistently sized. The disclosed diroximel fumarate particles are also characterized by a reduction in small particles (referred to as “fines”), as measured by D₁₀. A sample with a larger D₁₀ value will have fewer fine particles than a sample with a smaller D₁₀ value.

In a first embodiment, the disclosed diroximel fumarate particles have span of 1.1 to 2.5, a D₁₀ of 20-50 μm, a D₅₀ of 70-130 μm, and a D₉₀ of 150-250 μm. In a second embodiment, the diroximel fumarate particles have a span of 1.2 to 2.0, a D₁₀ of 20-50 μm, a D₅₀ of 70-130 μm, and a D₉₀ of 150-250 μm. In a third embodiment, the diroximel fumarate particles have a particle size span of 1.3 to 1.8, a D₁₀ of 20-50 μm, a D₅₀ of 70-130 μm, and a D₉₀ of 150-250 μm.

In a fourth embodiment, the disclosed diroximel fumarate particles have a D₁₀ of 30-45 μm, a D₅₀ of 80-120 μm, a D₉₀ of 155-230 μm and a span as described in the first, second or third embodiments. In a fifth embodiment, the disclosed diroximel fumarate particles have a D₁₀ of 36-42 μm, a D₅₀ of 88-113 μm, and a D₉₀ of 160-225 μm and a span as described in the first, second, or third embodiments.

The disclosed diroximel fumarate particles and the disclosed powder blends comprising the diroximel particles are characterized by superior flow properties, measured by the flow function coefficient and cohesion. Both flow function coefficient and cohesion are measured via shear testing. For the purpose of this application, “blend” and “powder blend” are synonymous.

“Flow function”, “flow function coefficient”, or FFc is a parameter commonly used to rank the flowability of a particulate sample, which could be a sample of neat API particles or a blend containing API particles plus additives.
Powder flowability is ranked as follows:
   FFc<1 not flowing
   1<FFc<2 very cohesive
   2<FFc<4 Cohesive
   4<FFc<10 easy flowing
   FFc>10 free flowing

The measurement of FFc and Cohesion outlined below follows the procedures described in ASTM D₆₇₇₃-16. An easy-flowing powder has an FFc of 4 or greater, and a free-flowing powder had an FFc of 10 or greater.

To test FFc and Cohesion, a particulate sample is added to a commercially available ring shear testing device, such as a Jenike-Johanson RST-XS ring shear tester. Such testing is familiar to the person of ordinary skill in the art. In general, the powder is loaded into an annular trough and a consolidated powder bed is formed. A normal stress (σp) is applied to the consolidated powder bed and the pre-shear stress (ζp) is measured. The normal stress is applied as a vertical load through an annular lid. The shear cell rotates relative to the lid and the torque necessary for shearing is measured. Repeat measurements with a series of values of normal stress are applied. When the measured shear stress values (Y-axis) are plotted as function of applied normal stress values (X-axis), the linear relation between them is called the yield locus. The yield locus line can be used to determine a unique relationship between normal stress and shear stress; this is known as a Mohr circle. The yield locus line represents a tangential line of all Mohr circles. The outer larger radius circle corresponds to pre-shear conditions (σp, ζp) and defines the Major Principal Stress, MPS (σ1). The smaller radius circle defines the Unconfined Yield Stress, UYS (σc).

Flow function coefficient or FFc is the ratio of Major Principle Stress (MPS) to the Unconfined Yield Strength (UYS):

In a sixth embodiment, the disclosed dioroximel fumarate particles (e.g., in embodiments one, two, three, four and five) have a flow function coefficient of between 3.5 and 20. In a seventh embodiment, the disclosed dioroximel fumarate particles (e.g., in embodiments one, two, three, four and five) have a flow function coefficient of between 4.0 and 20, between 4.0 and 10, between 4.0 and 4.5, or between 4 and 5.

Cohesion is the propensity for particles within a sample to self-associate or “stick together”. The ring shear tester measures cohesion at the same time that it measure FFc. The intercept of yield locus line at zero normal stress (i.e., where the extrapolation of the yield locus line intercepts the Y axis) is known as powder cohesion, and is expressed in Pascal (Pa) units. Lower cohesion is associated with better powder flowability. Further, cohesion predicts how particulate samples will behave when stored, for example when stored in a silo. A sample with a cohesion of greater than 250 Pa or greater is at risk of forming clumps and voids as the particles are too cohesive, whereas a sample with a cohesion of 250 Pa or less will generally remain an easy-flowing or free-flowing powder even after prolonged storage (e.g., one month to one year).

In an eighth embodiment, the disclosed dioroximel fumarate particles (e.g., in embodiments one, two, three, four, five, six, and seven) have a cohesion of 150 to 250 Pa, or 175 to 250 Pa, or 200 to 250 Pa, or 225 to 250 Pa, or 175 to 230 Pa, or 180 to 230 Pa or 200 to 225 Pa or 215 to 225 Pa.

In a ninth embodiment, the disclosed diroximel fumarate particles can be combined into a powder blend comprising one or more excipients, fillers and/or additives. A “blend” or a “powder blend” is a powder comprising particles of API with additives, fillers and/or excipients formulated for compression into tablets. The powder blends of the invention comprise the disclosed diroximel fumarate particles in the first, second, third, fourth, fifth, sixth, seventh, or eighth embodiments. Additives present in the disclosed powder blends include (but are not limited to) texturizers and anti-caking agents (e.g., microcrystalline cellulose), disintegrants (e.g., crospovidone), flow agents (e.g., colloidal silica), and release and lubricating agents (e.g., magnesium stearate). In a tenth embodiment, the powder blends comprise the diroximel fumarate particles of the first, second, third, fourth, or fifth embodiments and have a flow function coefficient of between 15 and 40. In an eleventh embodiment, the disclosed powder blends comprise the diroximel fumarate particles of the first, second, third, fourth, or fifth embodiments and have a flow function coefficient of between 17 and 37, 17 and 25, 18 and 22 or 19 and 21. In a twelfth embodiment, the disclosed powder blends comprise the diroximel fumarate particles of the first, second, third, fourth, or fifth embodiments and have a flow function coefficient of between 20 and 35 or 18 and 25.

In a thirteenth embodiment, the weight percent of disclosed diroximel fumarate particles in the powder blends described in the ninth, tenth, eleventh and twelfth embodiments is 87.5 wt. % based on the total weight of the powder blend. In a fourteenth embodiment, the weight percent of disclosed diroximel fumarate particles in the powder blends described in the ninth, tenth, eleventh and twelfth embodiments is 87-88 wt. % based on the total weight of the powder blend. In a twelfth embodiment, the weight percent of disclosed diroximel fumarate particles is 85-89 wt. % based on the total weight of the powder blend. In a fifteenth embodiment, the weight percent of disclosed diroximel fumarate particles in the powder blends described in the ninth, tenth, eleventh and twelfth embodiments is 84-90 wt. % based on the total weight of the powder blend. In a sixteenth embodiment, the weight percent of disclosed diroximel fumarate particles in the powder blends described in the ninth, tenth, eleventh and twelfth embodiments is 82-90 wt. % based on the total weight of the powder blend. In a seventeenth embodiment, the weight percent of disclosed diroximel fumarate particles in the powder blends described in the ninth, tenth, eleventh and twelfth embodiments is 60-92.5% wt. % (e.g., 70-92.5% wt. %. 75-92.5% wt. %, 80-92.5% wt. %, 85-92.5% wt. %, or 87.5 wt. %) based on the total weight of the powder blend.

In an eighteenth embodiment, the invention is a tablet comprising the disclosed diroximel fumarate particles in any one of embodiments 1-8; or the disclosed diroximel fumarate blends in any one of embodiments 9-17. The disclosed tablets are “high load”, i.e., they comprise 60%, 60-65%, 65%-70%, 70-75%, 75-85%, 85-87.5%, 87.5%, 87-88%, 85-89%, 84-90%, 82-90%, 70-92.5%, 80-90%, 60-92.5% or 70-92.5% by weight of the disclosed diroximel fumarate particles.

### Methods

Methods of producing the disclosed diroximel fumarate particles are included in the invention. These methods produce diroximel fumarate particles suitable for the preparation of high load tablets. The method comprises subjecting a slurry of pre-milled diroximel fumarate particles in a slurry solvent to a wet milling step to produce wet milled diroximel fumarate particles; and subjecting the wet milled diroximel fumarate particles to a ripening step in a ripening solvent to produce the disclosed diroximel fumarate particles. The milling and ripening step are conducted under conditions suitable for obtaining particles suitable for high load tableting, e.g., obtaining the disclosed diroximel fumarate particles. Suitable conditions include suitable shear and mixing for the milling step and suitable solvents, temperature and milling and ripening time periods. These conditions are described in greater detail below.

“Pre-milled diroximel fumarate particles” refers to diroximel fumarate particles before wet milling and ripening. Optionally, pre-milled diroximel fumarate particles are recrystallized prior to the wet milling step. For example, diroximel fumarate pre-milled may be dissolved in a sufficient amount of isopropyl acetate (such as 1:3 to 1:4 by weight) at a temperature in which the particles dissolved in the solvent (such as 70-80° C.). The solution is optionally filtered. The solution is then cooled to a suitable temperature to precipitate the pre-milled diroximel fumarate.

“Neat diroximel fumarate particles” refers to diroximel fumarate particles that have not had any additives intentionally added. Thus, “neat diroximel fumarate particles” could result from either dry milling or wet-milling and ripening methods of milling, and may be tested as neat particles prior to being incorporated into blends.

“Wet milling” refers to subjecting a slurry of diroximel fumarate particles in a slurry solvent to mixing at a suitable shear, mixing speed and temperature until a desired particle size reduction is achieved, for example a D₅₀ of from 35-70 μm. Alternatively, the desired particle size is between D₅₀ of 35-66 μm; 35-55 μm; 35-60 μm; or 55-66 μm. The slurry solvent and wet milling temperature are selected so that diroximel fumarate is substantially insoluble in the slurry solvent.

“Substantially insoluble” means that there is substantially no dissolution of diroximel fumarate in the slurry solvent at the milling temperature. Substantial insolubility includes a solubility of less than 44 mg/mL in the slurry solvent at the temperature at which the particles are milled, and preferably less than 28 mg/mL and even more preferably less than 22 mg/mL Isopropyl acetate is a suitable slurry solvent for pre-milled diroximel fumarate particles at wet milling temperatures between from −5° C. to 30° C. Other solvents that can be used include but are not limited to ethyl acetate, methyl ethyl ketone, isobutyl acetate, toluene, and acetone; and combination of one or more solvents with an antisolvent such as: water, 1-butanol, 3-methyl-1-butanol, 2-butanol, 1-pentanol, 1-propanol, 2-propanol, ethanol, heptane, t-butanol, acetonitrile, and methyl tert-butyl ether.

The wet milling step comprises subjecting a slurry of diroximel fumarate particles in a slurry solvent to mixing at a shear from 11,500 s⁻¹ to 160,000 s⁻¹ at a mixing speed from 600 to 1500 revolutions per minute (RPM). Alternatively, the wet milling step comprises subjecting a slurry of diroximel fumarate particles in a slurry solvent to mixing at a shear from 11,500 s⁻¹ to 100,000 s⁻¹ at a mixing speed from 1000 to 1300 revolutions per minute (RPM) at a temperature from 0° C. to 20° C. Alternatively, the wet milling step comprises subjecting a slurry of diroximel fumarate particles in a slurry solvent to mixing at a shear from 11,500 s⁻¹ to 25,000 s⁻¹ at a mixing speed from 1000 to 1300 revolutions per minute (RPM). Wet milling can be performed using a commercially available high-shear mixer, e.g., an inline high-shear mixer (e.g., Silverson Verso inline mixer or IKA MagicLab inline mixer).

The wet milling step can be carried out at a temperature from −5° C. to 30° C. In another aspect, the wet milling step is carried out at a temperature from −5° C. to 15° C., 15° C. to 25° C., or 25° C. to 30° C.

“Ripening” refers to heating the milled diroximel fumarates particle in a ripening solvent in which diroximel fumarate is partially soluble at the temperature at which the milled particles are ripened. “Partially soluble” means that there is a partial dissolution and then re-precipitation of the diroximel fumarate particles in the solvent at the temperature at which the ripening occurs. Partial solubility includes a solubility of between 55 mg/mL and 107 mg/mL in the ripening solvent at the temperature at which the particles are ripened. Isopropyl acetate is a suitable solvent for the ripening process at temperatures between 35° C. to 50° C. alternatively 38° C. to 49° C., and, in another alternative, between 40° C. to 48° C. Other solvents that can be used include but are not limited to ethyl acetate, methyl ethyl ketone, isobutyl acetate, toluene, and acetone; and combination of one or more solvents with an antisolvent such as: water, 1-butanol, 3-methyl-1-butanol, 2-butanol, 1-pentanol, 1-propanol, 2-propanol, ethanol, heptane, t-butanol, acetonitrile, and methyl tert-butyl ether. Ripening leads to an increase in particle size and a decrease in particle size distribution (e.g., span). The ripening process is continued until the desired particle size is achieved, e.g., a D₅₀ of 70-130 μm. Alternatively, the desired particle size is a D₅₀ of 70-130 μm. In another alternative, the desired particle size is a D₅₀ 88-113 μm. Suitable time periods for ripening include from two to ten hours, or from two to eight hours, or from two to four hours.

The method optionally comprising a cooling step after the ripening step. After the ripening step, the mixture can be cooled to between −10° C. to 20° C., alternatively to between −5° C. to 10° C. The diroximel fumarate particles are then isolated.

The method is illustrated by the following examples, which are not intended to be limiting.

### EXEMPLIFICATION

### Example 1: Comparative Dry Milled Particles

Diroximel fumarate particles were processed using a conventional Hammermill dry mill with a hammer tip speed ranging from 47 to 62 m/s (3500-6000 RPM) and a feed rate ranging from 1.4 to 7.2 kg/min. The relatively large span of 2.9-3.4 indicates that the particles within each sample lack uniformity of size. Particle size distribution data for four commercial lots of the dry milled particles is shown in Table 1.

TABLE 1
Particle Size Distribution of dry milled
diroximel fumarate API (in micrometers)

| Entry No. | | | | |
|---|---|---|---|---|
| (representative | | | | |
| commercial lot) | D₁₀ (μm) | D₅₀ (μm) | D₉₀ (μm) | Span |
| 1 | 17 | 96 | 347 | 3.4 |
| 2 | 17 | 106 | 347 | 3.1 |
| 3 | 18 | 101 | 307 | 2.9 |
| 4 | 16 | 95 | 302 | 3.0 |

Note:
Span is calculated as (D₉₀ − D₁₀)/D₅₀, is a measured of particle size distribution around the mean D₅₀.

### Example 2: Wet Milled and Ripened Particles

507 kg diroximel fumarate API was dissolved in 2771 kg isopropyl acetate at 80° C. and polished filtered. The solution was then cooled to 40° C. in about 3 hours (h) to partially crystallize the diroximel fumarate then reheated to 55° C. and held at that temperature for 30 min followed by cooling to 00.

After a period of stirring, the slurry was recirculated from the reactor bottom through a high-shear mixer (IKA type DR2000-30 available from IKA, Staufen, Germany) and back to the reactor. The mixer was operated at 1000-1300 RPM and with shear values within 11,500 s-1 to 25,000 s-1 while maintaining the slurry temperature not to exceed 200. The total mass or volume of slurry passed through the mixer was monitored by mass flow meter and the mill was stopped after at least 20 volume turnovers. The milling was considered completed when the median particle size was within 35-60 microns. PSD data are shown in Table 2 below as “PSD after wet milling.”

After the milling step was completed, 500 kg isopropyl acetate was added to rinse the recirculation line and was combined with the milled slurry in the reactor. The resulting slurry was ripened at a selected temperature within 38-50° C. in not less than 2 h, held for 10-30 min and cooled back to 0° C. in 2-8 h. The product was filtered, washed with 439 kg isopropyl acetate and dried under vacuum at 40-50° C. PSD data are shown in Table 2 below as “Final PSD”.

TABLE 2
Particle Size Distribution of wet milled and ripened diroximel fumarate API
(in micrometers)

| | | | | | Ripening | | | | |
|---|---|---|---|---|---|---|---|---|---|
| | Batchx | PSD after wet milling | | | Temper- | Final PSD after ripening | | | |
| Entry | size | D₁₀ | D₅₀ | D₉₀ | ature | D₁₀ | D₅₀ | D₉₀ | |
| No | (kg) | (μm) | (μm) | (μm) | (° C.) | (μm) | (μm) | (μm) | Span |
| 1 | 507 | 11.6 | 53.0 | 155 | 40 | 37.2 | 89.3 | 162.5 | 1.4 |
| 2 | 507 | 12.4 | 66.3 | 179 | 43 | 40.7 | 96.1 | 204.5 | 1.7 |
| 3 | 507 | 9.15 | 35.2 | 116 | 48 | 41.0 | 111.9 | 222.9 | 1.6 |

Note:
Span is calculated as (D₉₀ − D₁₀)/D₅₀, is a measured of distribution tightness around the mean d50.

By comparing the particle sizes and particle size distribution (PSD) of comparative dry milled diroximel fumarate in Example 1, it is clear that the D₁₀ (values: 16-18 μm) are lower for the comparative particles of Example 1 compared to the higher D₁₀ (values: 37.2-41.0 μm) for the wet milled and ripened particles. Further, the span is higher for the comparative particles of Example 1 (values: 2.9-3.4) compared to the lower span (values: 1.4-1.7) for the wet milled and ripened particles. Thus, the wet milled and ripened particles are expected to have better tablet forming properties than the dry-milled particles.

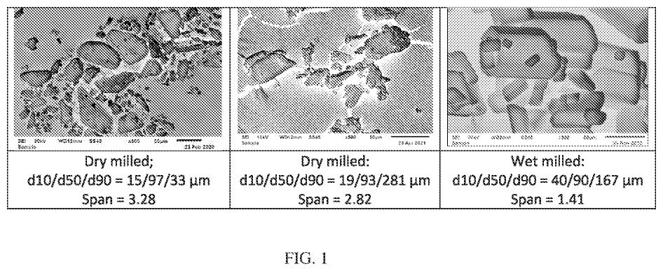

Scanning electron microscopy was used to show particles from dry milling vs. wet milling in FIG. 1. The images were collected using a Scanning Electron Microscope (SEM) (JEOL USA Inc. Peabody, MA Model JSM-6510LV] at 350× to 500× magnification. Notice the presence of particle fines in the two dry milled lots of diroximel fumarate (pictured left and center); these lots also have particles with highly irregular shapes. In contrast, the wet milled batch of diroximel fumarate (pictured right) has few particle fines and the particles have regular, flattened cuboid shaped.

### Example 3: Comparison of Flow Function Coefficient and Cohesion of Neat Diroximel Fumarate Particles

The flow function coefficient (FFc) was measured as described above to quantify the flow properties of neat diroximel fumarate particles of Example 1 (dry milled) and Example 2 (wet milled and ripened). The particles were tested in a Jenike-Johanson RST-XS Ring Shear Tester according to the manufacturer's procedure and using the software provided with the tester. Each particulate or powder sample was loaded (one at a time) into the annular trough of the tester. A pre-load force of 3 kPa was first applied to the powder blend. Subsequent loads ranging from 0.5 kPa to 3 kPa were then applied to determine the flow properties of each sample. The Ring Shear Tester computes yield locus line, the FFc (i.e., the slope of the yield locus line), and the cohesion (i.e., the Y-intercept of the extrapolated yield locus line)

As a person of ordinary skill will understand, the FFc and cohesion will vary from lot to lot of particles; thus, it is important to look both at individual lot values as well as the uniformity or tightness of the measurements taken from different lots of material that has been processed by any given method.

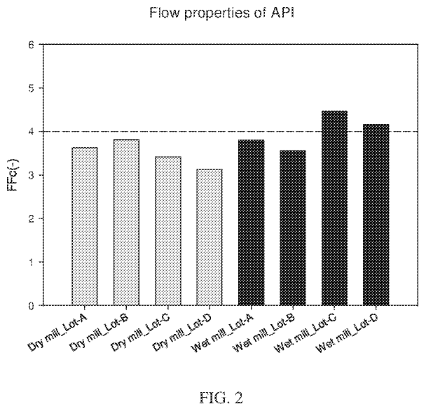

FIG. 2 is a bar graph comparing the FFc of four lots of neat diroximel fumarate particles made with the comparative neat dry milled diroximel fumarate of Example 1 (gray bars) with four lots of neat wet milled and ripened diroximel fumarate of Example 2 (black bars). The four wet milled lots had an average FFc of about 4, compared to an average FFc of less than 4 for the four dry milled lots. An FFc of 4 is significant, as 4 is the threshold value for an easy-flowing powder range, as described above in the detailed description. Thus, the wet milled neat diroximel fumarate particles have improved flowability over the comparative dry milled neat diroximel fumarate particle. More importantly, the disclosed particles are classified as free-flowing powders with an FFc of 4 or greater.

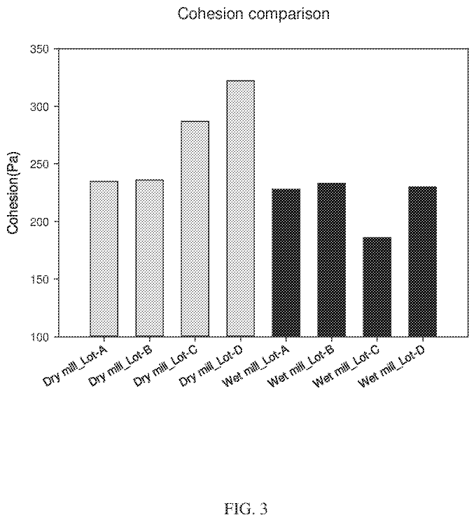

The comparison of cohesion for four lots of neat diroximel fumarate particles obtained from hammer mill dry process diroximel fumarate particles versus four lots of neat wet milled and ripened diroximel fumarate particles are depicted in FIG. 3. The lower the cohesion the better the sample flows relative to samples having higher Cohesion. The wet milled particles (black bars) have an average cohesion of 219 Pa, and are thus less cohesive than dry milled particles (gray bars) that have and average cohesion of 270 Pa. Less cohesion of the diroximel fumarate particles leads to better compaction properties of blends comprising the particles and to better storage properties. Further, the variation between lots was less for the inventive lots versus the comparative lots.

### Example 4: Comparison of Flow Function Coefficient of Powder Blends with High Load of Particles

The wet milled and ripened diroximel fumarate particles produced in Example 2 were used to make four lots of the powder blend of Table 3. Four lots containing the comparative dry milled particles from Example 1 were also prepared.

TABLE 3
Powder Blend composition

| | Source | Amount | |
|---|---|---|---|
| Diroximel fumarate | Example 2 | 81-92.5% | w/w |
| Microcrystalline | DuPont Nutrition, | 3-6% | w/w |
| cellulose | | | |
| Avicel 102 | Ireland | | |
| crospovidone/Kollidon C | BASF, Germany | 3-6% | w/w |
| Colloidal silica 200 | Evonik, NJ, USA | 1-3% | w/w |
| Magnesium stearate | Spectrum Chemical MFG. | 0.5-2% | w/w |
| | Corp. NJ, USA | | |

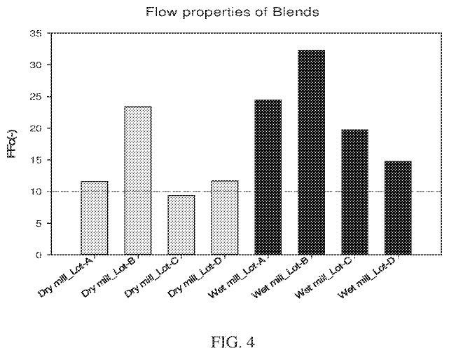

The flow function coefficient was also used to quantify the flow properties for the powder blends made according to Table 3. FIG. 4 is a bar graph comparing the FFc of four lots of blend according to Table 3 made with the comparative dry milled diroximel fumarate of Example 1 (gray bars) with the FFc four lots of blend containing wet milled and ripened diroximel fumarate of Example 2 (black bars). As stated above, an FFc value greater than 10 indicates a free flowing powder. The wet milled and ripened diroximel fumarate blends consistently exhibit higher FFc (with an average FFc value of around 20) than the dry milled diroximel fumarate blends (with an average FFc value of around 15, which means the blends containing the wet-milled particles have better flow properties compared to otherwise identical blends containing the dry milled diroximel fumarate, as evidenced by the improvement in FFc of from about 15 to about 20.

### Example 5: Tablet Compaction Profiles

The formulated the powder blends were compressed at different compaction forces (4-20 kN) on compaction simulator (Romaco Kilian STYL'ONE, Cologne, Germany); the simulated tablet press is GEA-Courtoy Modul P. A 16-tip of tooling with 2 mm concave punch (Natoli Engineering Company, USA) was employed. The tableting speed was set at 20 rpm, the target tablet weight was 8.5 (+0.5) mg. All the prepared mini-tablets were stored in a sealed plastic bag for further characterizations.

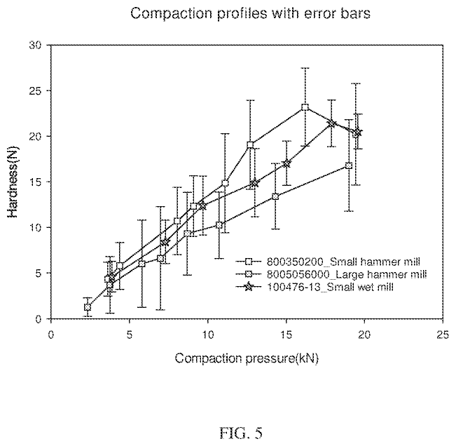

The tablet hardness was measured under a diametrical compression test in a texture analyzer (Texture Technologies Corp., USA). The average tablet hardness was recorded (n=10). The comparison of compaction profiles (hardness vs compaction pressure) for tablets made using diroximel fumarate particles obtained from hammer mill dry process lots versus wet milled and ripened lots are depicted in FIG. 5. The dry mill lots (squares) and the wet mill lot (stars) showed comparable compaction profiles at lower compaction pressure (compaction pressure lower than 10 kN), which means they have similar tablet hardness at the same compaction pressure. However, the error bars of the wet milled lot is much smaller compared to both hammer mill lots. This indicates that the tablet hardness is more uniform than the tablets made by dry milling; the more uniform hardness is beneficial for the tablet coating process as well as dissolution behavior of the pressed tablet.

### Example 6: Wet Milled and Ripened Particles in a Solvent Mixture Isopropyl Acetate (IPAC) and Isopropyl Alcohol (IPA)

55.2 kg of diroximel fumarate was dissolved in solvent mixture consisting of 137.4 kg IPAC and 183.3 kg IPA at 75-85° C. After complete dissolution of the diroximel fumarate the mixture was cooled to 0-5° C. over 8 hours. The resulting slurry of diroximel fumarate was then subjected to wet milling by recirculating the slurry through an IKA high-shear mixer type DR-2000-10 operated at 2600-4200 rpm (shear values within 30,000-50,000 sec-1). The recirculation was stopped after 23 volume turnovers, after which an in-process measurement showed the median particle size was reduced to D₅₀₌₄₃ microns. A 45.8 kg mixture of IPA/IPac (6:4 v/v) was added as line rinse. The combined line rinse and reactor contents were stirred at 0-5° C. for 1 hour followed by ripening with the following conditions: heated to 43° C. in 2 h, held at 43° C. for 2 h, cooled to 2° C. in 3-5 h and stirred for an additional 2 h at 0-5° C. The product was filtered and washed with a 45.8 kg mixture of IPA/IPAc (6:4 v/v) and dried under vacuum at 35-45° C. for 20-28 h to yield 50.2 kg wet milled diroximel fumarate product with the following particle size distribution: D₁₀=40 μm, D₅₀=92 μm, and D₉₀=173 μm.

By comparing the particle sizes and particle size distribution (PSD) of comparative dry milled diroximel fumarate in Example 1, it is clear that the D₁₀ (values: 16-18 μm) are lower for the comparative particles of Example 1 compared to the higher D₁₀ (values 40 μm) for the wet milled and ripened particles of this example. Further, the span is higher for the comparative particles of Example 1 (values: 2.9-3.4) compared to the lower span (value: 1.4) for the wet milled and ripened particles of this example. Thus, the wet milled and ripened particles are expected to have better tablet forming properties than the dry-milled particles.

### Example 7: Comparison Dissolution Performance

The formulated powder blends were compressed into mini tablets using a two-sided tablet press (Fette Compacting GmbH, Germany), equipped with multi-tip tooling (Natoli Engineering Company, USA). The mini tablets were dedusted and passed through a metal detector. The complete mini tablets were held in bins and the sealed bin was staged for tablet coating. For the enteric coating, the coating solution was prepared in two separate components: a solution of the coating polymer, Eudragit with the plasticizer, Triethyl Citrate (TEC) was prepared in approximately half of the 60:40 Isopropyl Alcohol (IPA)/Water mixture. Talc and Colloidal Silicon Dioxide were suspended in the remaining half of 60:40 Isopropyl Alcohol (IPA)/Water mixture. The two solution components were mixed together to form the enteric coating suspension. At the completion of the pre-heat phase, a solvent based enteric coat was applied to the tablet core to achieve a theoretical weight gain 15% relative to the mass of the core. IPA and water was removed during the film coating process. This application of an enteric coat was performed to control the release of the API within the gastro-intestinal tract.

The coated mini-tablets (33 tablets) were encapsulated into size 0 capsule. The capsule was inserted into a probe sinker, 750 mL of degassed 0.1 M HCl medium were added into each vessel, which were then heated to 37° C.±0.5° C. for acid stage dissolution testing. After two hours, another 200 mL of the pre-heated 0.2M sodium phosphate tribasic dissolution medium were added into each dissolution vessel for buffer stage dissolution testing. The HPLC (Waters Corp, USA) was used for dissolution analysis.

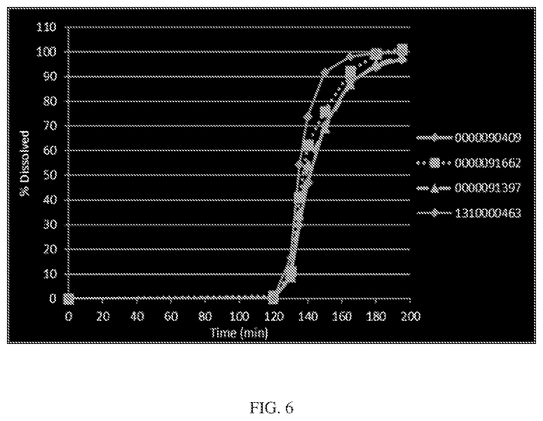

The comparison of dissolution profiles for tablets made using diroximel fumarate particles obtained from hammer mill dry process lots versus wet milled and ripened lot are depicted in FIG. 6. The left-most red diamond plot curve (Wet milled) and blue diamond, square and triangle plot curves (Dry milled) showed comparable dissolution behavior at acid stage (0-12 0 min). However, the dissolution (120-200 min) is much faster for tablets made from wet milled API lots compared to all dry milled lots. It indicates that the wet milled API particles have better dissolution performance when used in drug product. This faster dissolution behavior could be attributed to uniform particle size distributions, smooth surface, and uniform coating.

TABLE 4
Particle properties of diroximel fumarate particle lots tested in Example 7.

| DP Lot | 1310000447 | 1310000448 | 1310000449 | 1310000463 |
|---|---|---|---|---|
| API milling | Dry mill | Dry mill | Dry mill | Wet mill |
| process | | | | |
| API Lot | 8005663000 | 8005685000 | 8005684000 | 21600T0003 |
| PSD D10 | 17 | 17 | 18 | 40.7 |
| (μm) | | | | |
| PSD D50 | 91 | 91 | 98 | 96.1 |
| (μm) | | | | |
| PSD D90 | 255 | 270 | 279 | 205 |
| (μm) | | | | |
| PSD Span | 2.62 | 2.78 | 2.66 | 1.71 |

As demonstrated in the leftmost curve in FIG. 6, the wet-milled diroximel fumarate particles showed greater than 50% dissolution at 130 minutes, greater than 70% dissolution at 140 minutes, greater than 90% dissolution at 150 minutes, and nearly 100% dissolution by 160 minutes.

What is claimed is:

1. Diroximel fumarate particles having a span of 1.1 to 2.5, a D₁₀ of 20-50 μm, a D₅₀ of 70-130 μm, and a D₉₀ of 150-250 μm.

2. The diroximel fumarate particles of claim 1, wherein the span is 1.2 to 2.0.

3. The diroximel fumarate particles of claim 1, wherein the span is 1.3 to 1.8.

4. The diroximel fumarate particles of any one of claims 1-3, having a D₁₀ of 30-45 μm, a D₅₀ of 80-120 μm, and a D₉₀ of 155-230 μm.

5. The diroximel fumarate particles of any one of claims 1-3, having a D₁₀ of 36-42 μm, a D₅₀ of 88-113 μm, and a D₉₀ of 160-225 μm.

6. The diroximel fumarate particles of any one of claims 1-5 having a flow function coefficient of between 4 and 20.

7. The diroximel fumarate particles of any one of claims 1-5 having a cohesion of between 150 and 250 Pascal (Pa).

8. A blend, comprising the diroximel fumarate particles of any one of claims 1-5 and one or more additives, fillers, and/or excipients and wherein the blend has a flow function coefficient of between 15 and 40.

9. A blend, comprising the diroximel fumarate particles of any one of claims 1-5 and one or more additives, fillers, and/or excipients and wherein the blend has a flow function coefficient of between 17 and 37.

10. The blend of claim 8 or 9, wherein the blend comprises from 60-92.5 wt. % diroximel fumarate particles based on the total weight of the powder blend.

11. The blend of any one of claims 8-10, wherein the additives, fillers, or excipients are microcrystalline cellulose, crospovidone, colloidal silica magnesium stearate.

12. A tablet comprising the diroximel fumarate particles of any one of claims 1-7 or the powder blend of any one of claims 8-11.

13. A method of producing diroximel fumarate particles suitable for use in high load tablets, comprising:
subjecting a slurry of pre-milled diroximel fumarate particles in a slurry solvent to a wet milling step to produce milled diroximel fumarate particles; and
subjecting the milled diroximel fumarate particles to a ripening step in a ripening solvent to produce the diroximel fumarate particles.

14. The method of claim 13, wherein the pre-milled diroximel fumarate particles are substantially insoluble in the slurry solvent at the temperature at which the pre-milled diroximel particles are milled.

15. The method of claim 13 or 14, wherein the wet milling comprises subjecting the slurry of pre-milled diroximel fumarate particles in the slurry solvent to mixing at a shear from 11,500 s⁻¹ to 160,000 s⁻¹ at a mixing speed from 600 to 1500 revolutions per minute (RPM).

16. The method of claim 15, wherein the wet milling step comprises subjecting the slurry of pre-milled diroximel fumarate particles in the slurry solvent to mixing at a shear from 11,500 s⁻¹ to 25,000 s⁻¹ at a mixing speed from 1000 to 1300 revolutions per minute (RPM).

17. The method of any one of claims 13-16, wherein the slurry solvent is isopropyl acetate.

18. The method of any one of claims 13-17, wherein the wet milling step is carried out at a temperature from 0° C. to 20° C.

19. The method of any one of claims 13-18, wherein the wet milling step is continued until the wet milled diroximel fumarate particles achieve a D₅₀ of 35-70 μm.

20. The method of any one of claims 13-18, wherein the wet milling step is continued until the wet milled diroximel fumarate particles achieve a D₅₀ of 35-60 μm.

21. The method of any one of claims 13-20, wherein the ripening step comprises heating the wet milled diroximel fumarate particles in a ripening solvent in which the wet milled diroximel fumarate particles are partially soluble at the temperature at which the wet milled diroximel fumarate particles are ripened.

22. The method of claim 21, wherein the ripening solvent is isopropyl acetate.

23. The method of any one of claims 13-22, wherein the temperature at which the wet milled diroximel fumarate particles are ripened is from 38° C. to 50° C.

24. The method of any one of claims 13-22, wherein the temperature at which the wet milled diroximel fumarate particles are ripened is from 40° C. to 48° C.

25. The method of any one of claims 13-24, wherein the ripening step is continued until the ripened diroximel fumarate particles achieve a D₅₀ of 70-130 μm.

26. The method of any one of claims 13-25, wherein the ripening step is conducted for 2 to 10 hours.

27. The method of any one of claims 13-26, wherein the pre-milled diroximel fumarate is recrystallized prior to the wet milling step.

28. The method of any one of claims 13-27, further comprising a cooling step after the ripening step.

29. The method of claim 28, wherein the temperature of the cooling step is −10° C. to 10° C.

30. Diroximel fumarate particles produced by the method of any one of claims 13-29.
